(19) United States
(12) Patent Application Publication
(10) Pub. No.: US 2024/0157074 A1
(43) Pub. Date: May 16, 2024

(54) AUTOMATED OXYGEN THERAPY DEVICE AND RELATED METHODS

(71) Applicant: MyAutO2, LLC, Seattle, WA (US)

(72) Inventors: Stephen Harry Goldberg, Seattle, WA (US); Kendrick Lin Hougen, Indianapolis, IN (US); Dana Michelle Lewis, Woodinville, WA (US); Scott Michael Leibrand, Woodinville, WA (US); Arthur Stewart Kerr, Lowell, MA (US); Jonathon Wayne Lough, Highlands Ranch, CO (US); Alexander Jon Erikson, Ann Arbor, MI (US)

(21) Appl. No.: 18/552,415

(22) PCT Filed: Mar. 18, 2022
(86) PCT No.: PCT/US22/20939

§ 371 Date: Sep. 25, 2023

Related U.S. Application Data
(60) Provisional application No. 63/166,453, filed on Mar. 26, 2021.

(51) Int. Cl. A61M 16/00

(52) U.S. Cl. CPC A61M 16/026; A61M 16/0051; A61M 2016/003; A61M 2202/0007; A61M 2202/0208; A61M 2205/3303; A61M 2205/3334; A61M 2205/50; A61M 2230/005; A61M 2230/04; A61M 2230/20; A61M 2230/42; A61M 2230/43; A61M 2230/63

(57) ABSTRACT

A device for administering oxygen therapy to a patient comprises a gas intake valve; a primary flow path connecting the gas intake valve to a gas output connector, the primary flow path comprising a flow controller configured to adjust a first flow rate through the primary flow path, wherein the flow controller is in electronic communication with a processor and memory; at least one physiological sensor communicatively coupled to the processor performing a method comprising: receiving a target oxygen saturation level; receiving at least one measured physiological parameter from the at least one physiological sensor; analyzing the at least one measured physiological parameter to determine a predicted oxygen saturation level of a patient for a first time window; and adjusting the first flow rate with the flow controller to bring the predicted oxygen saturation level within a threshold value of the target oxygen saturation level.

### CROSS-REFERENCE TO RELATED APPLICATION

This application claims the priority benefit of U.S. Provisional Application No. 63/166,453, filed on Mar. 26, 2021, the disclosure of which is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of medical equipment and, more specifically, to the field of medical gas delivery systems. Described herein are systems and methods for the administration of oxygen therapy to a patient.

### BACKGROUND

Oxygen therapy, also known as oxygen titration, is a mainstay treatment for a variety of conditions. In the hospital setting, medical grade oxygen is provided to the patient with a nose cannula or similar device through a wall-mounted system with a hospital supply of gas or through individual supply tanks. To adjust the flow rate of gas to the patient, a medical practitioner or a patient during a home use arrangement, must manually operate physical or digital controls. Although various medical sensors can be fitted to a patient and can inform an adjustment of the flow rate, these simple systems require human interaction to execute a change. As a patient's need for medical oxygen can change fairly quickly according to a wide variety of patient specific and situational conditions, this requirement for manual control can become a barrier to consistently administering the correct therapeutic dose. With many hospitals and homecare arrangements lacking sufficient resources to provide rigorous personal monitoring of a patient's oxygen supply, such systems can sometimes result in either too high of a flow rate (therefore wasting valuable medical supplies) or too low (exposing the patient to at least discomfort if not actual harm). Liberal oxygen administration leads to increased rates of hospital-acquired pneumonia and infections, decreased respiratory drive in chronically hypercarbic patients, vasoconstriction with reduced downstream perfusion of vital tissue such as cerebral and coronary structures, and systemic effects of oxygen toxicity. Hypoxemia, or low blood oxygen levels, can also be particularly detrimental in hospitalized patients, causing poor wound healing, reduced end organ perfusion, and central nervous system dysfunction. There remains a clear and present need for an adaptive and automated oxygen therapy system that allows for an automated adjustment of the flow rate according to the patient's fluctuating physiological status and need.

### SUMMARY

There is a need for new and useful system and method for administering oxygen therapy to a patient. In particular, there is a need for systems, devices, and methods that analyze measured physiological parameters to predict oxygen saturation levels for a patient and trigger adjustments of flow rates via flow controllers to bring a predicted oxygen saturation level within a threshold value of the target oxygen saturation level.

In various embodiments, the disclosure herein includes a device for administering oxygen therapy to a patient, the device comprising: a gas intake valve, a primary flow path connecting the gas intake valve to a gas output connector, the primary flow path comprising a flow controller configured to adjust a first flow rate through the primary flow path, wherein the flow controller is in electronic communication with a processor and memory, at least one physiological sensor communicatively coupled to the processor, wherein the memory stores machine-readable instructions that, when executed by the processor, cause the processor to perform a method comprising: receiving a target oxygen saturation level, receiving at least one measured physiological parameter from the at least one physiological sensor, analyzing the at least one measured physiological parameter to determine a predicted oxygen saturation level of a patient for a first time window, and adjusting, based on the analyzing of the at least one physiological parameter, the first flow rate with the flow controller to bring the predicted oxygen saturation level within a threshold value of the target oxygen saturation level.

In some embodiments, the device further comprises a bypass flow path connecting the gas intake valve to the gas output connector, wherein the bypass flow path comprises a manual flow rate adjuster. In some embodiments, the primary flow path and the bypass flow path comprise a flowmeter. In other embodiments, the primary flow path and the bypass flow path comprise a shared flowmeter, and wherein the primary flow path and the bypass flow path merge upstream of the shared flowmeter. In some embodiments, the device is configured to deliver gas through the primary flow path under a first operating condition and through the bypass flow path under a second operating condition, wherein the device is configured to change from the first operating condition to the second operating condition when a bypass criterion is detected or inputted.

In some embodiments, the device further comprises a bypass switch, wherein the bypass criterion is selected from the group consisting of: a loss of device power, a system error, or a user selection of the emergency bypass switch. In some embodiments, the device is configured to change from the second operating condition to the first operating condition by a user input.

In some embodiments, the oxygen intake valve is a solenoid valve. In some embodiments, the manual flow rate adjuster is a gas regulator. In some embodiments, the flowmeter is a Thorpe tube flowmeter. In some embodiments, the threshold value is about 5% to about 0.5% of the target oxygen saturation level. In other embodiments, the threshold value is about 0.5% of the target oxygen saturation level.

In some embodiments, the at least one physiological sensor is selected from the group consisting of: an SpO2 sensor, an EtCO2 sensor, an accelerometer, a pressure sensor, a microphone, a heart rate monitor, a back pressure monitor, an SpO2 waveform monitor, and an EKG. In further embodiments, the at least one measured physiological parameter is selected from the group consisting of: an oxygen saturation level, a rate of change of an oxygen saturation level, an EtCO2 level, a rate of change of an EtCO2 level, a respiratory rate, a rate of change of a respiratory rate, a heart rate, a rate of change of a heart rate, a motion of the patient, an activity level of the patient an audio signal of a patient breathing, a cyclic back pressure, and a chest rise monitoring.

In some embodiments, the device further comprises at least one internal sensor. In further embodiments, the at least one internal sensor is selected from the group consisting of a humidity sensor and a pressure sensor.

In some embodiments, the first time window is about 1 second to about 5 hours. In other embodiments, the first time window is about 1 second to about 2.5 hours. In still other embodiments, the first time window is about 1 second to about 1 hour. In further embodiments, the first time window is about 1 minute to about 1 hour. In still further embodiments, the first time window is about 1 minute to about 10 minutes. In additional embodiments, the first time window is about 1 minute to about 5 minutes. In still additional embodiments, the first time window is about 5 minutes to about 1 hour. In further additional embodiments, wherein the first time window is about 5 minutes to about 30 minutes.

In some embodiments, receiving the minimum oxygen saturation level further comprises receiving a user input of the minimum oxygen saturation level. In other embodiments, receiving the target oxygen saturation level further comprises receiving a user input of the target oxygen saturation level. In further embodiments, receiving the target oxygen saturation level further comprises: receiving one or more user inputs, and determining the target oxygen saturation level based on the one or more user inputs.

In some embodiments, the method performed by processor further comprises decreasing at least one of the target oxygen saturation level or first flow rate over a second time window down to an unassisted physiological level.

In some embodiments, the device further comprises an alarm, wherein the at least one measured physiological parameter comprises a measured oxygen saturation level, and wherein the method further comprises: comparing the measured oxygen saturation level to the minimum oxygen saturation level, and triggering the alarm when the measured oxygen saturation level is less than the minimum oxygen saturation.

In some embodiments, the device further comprises an alarm and wherein the method further comprises: measuring a first respiratory rate with at least one usage dependent sensor, measuring a second respiratory rate with at least one usage independent sensor, comparing the first respiratory rate with the second respiratory rate, and wherein when the first respiratory rate and second respiratory rate are not within a respiratory comparison threshold value, triggering at least one of an alarm or an instruction condition.

In various embodiments, the disclosure herein includes for a device for administering oxygen therapy to a patient, the device comprising: a gas intake valve, a primary flow path connecting the gas intake valve to a gas output connector, the primary flow path comprising a flow controller configured to adjust a first flow rate through the primary flow path, wherein the flow controller is in electronic communication with a processor and memory, a bypass flow path connecting the gas intake valve to the gas output connector, wherein the bypass flow path comprises a manual flow rate adjuster, at least one flow meter in fluid communication with and downstream of the flow controller and the manual flow rate adjuster, at least one physiological sensor communicatively coupled to the processor, at least one auditory or visual alarm, wherein the processor and memory store machine-readable instructions that, when executed by the processor, cause the processor to perform a method comprising: receiving a target oxygen saturation level, receiving a minimum oxygen saturation level, receiving at least one measured physiological parameter from the at least one physiological sensor, wherein the at least one measured physiological parameter comprises a measured oxygen saturation level, analyzing the at least one measured physiological parameter to determine a predicted oxygen saturation level of a patient for a first time window, comparing the measured oxygen saturation level to the minimum oxygen saturation level and triggering the alarm when the measured oxygen saturation level is less than the minimum oxygen saturation, and adjusting the first flow rate with the flow controller to bring the predicted oxygen saturation level within a threshold value of the target oxygen saturation level, wherein the device is configured to deliver oxygen gas through the primary flow path under a first operating condition, wherein the device is configured to deliver oxygen gas through the bypass flow path under a second operating condition, and wherein the device is configured to change from the first operating condition to the second operating condition when an emergency criterion is detected or inputted.

In various embodiments, the disclosure herein includes for a method for delivering oxygen therapy to a patient comprising: providing a device comprising: a gas intake valve, a primary flow path connecting the gas intake valve to a gas output connector, the primary flow path comprising a flow controller configured to adjust a first flow rate through the primary flow path, and at least one physiological sensor, receiving a target oxygen saturation level, receiving at least one measured physiological parameter from the at least one physiological sensor, analyzing the at least one measured physiological parameter to determine a predicted oxygen saturation level of a patient for a first time window, adjusting the first flow rate with the flow controller to bring the predicted oxygen saturation level within a threshold value of the target oxygen saturation level.

In some embodiments, the threshold value is about 5% to about 0.5% of the target oxygen saturation level. In other embodiments, the threshold value is about 0.5% of the target oxygen saturation level.

In some embodiments, at least one of the target oxygen saturation level or first flow rate is decreased over a second time window down to an unassisted physiological level.

In some embodiments, the at least one physiological sensor is selected from the group consisting of: an SpO2 sensor, an EtCO2 sensor, an accelerometer, a pressure sensor, a microphone, a heart rate monitor, a back pressure monitor, an SpO2 waveform monitor, and an EKG. In further embodiments, the at least one measured physiological parameter is selected from the group consisting of: an oxygen saturation level, a rate of change of an oxygen saturation level, an EtCO2 level, a rate of change of an EtCO2 level, a respiratory rate, a rate of change of a respiratory rate, a heart rate, a rate of change of a heart rate, a motion of the patient, an activity level of the patient, an audio signal of a patient breathing, a cyclic back pressure, and a chest rise monitoring.

In some embodiments, the device further comprises an alarm, wherein the at least one physiological parameter comprises a measured oxygen saturation level and wherein the method further comprises: receiving a minimum oxygen saturation level, comparing the measured oxygen saturation level to the minimum oxygen saturation level, and triggering the alarm when the measured oxygen saturation level is less than the minimum oxygen saturation.

In some embodiments, the first time window is about 1 second to about 5 hours. In other embodiments, the first time window is about 1 second to about 2.5 hours. In still other embodiments, the first time window is about 1 second to about 1 hour. In further embodiments, the first time window is about 1 minute to about 1 hour. In still further embodiments, the first time window is about 1 minute to about 10 minutes. In additional embodiments, the first time window is about 1 minute to about 5 minutes. In still additional embodiments, the first time window is about 5 minutes to about 1 hour. In further additional embodiments, the first time window is about 5 minutes to about 30 minutes.

In some embodiments, receiving the target oxygen saturation level further comprises receiving a user input of the target oxygen saturation level. In further embodiments, receiving the minimum oxygen saturation level further comprises receiving a user input of the minimum oxygen saturation level. In other embodiments, receiving the target oxygen saturation level further comprises: receiving one or more user inputs, and determining the target oxygen saturation level based on the one or more user inputs.

In some embodiments, the device further comprises an alarm and wherein the method further comprises: measuring a first respiratory rate with at least one usage dependent sensor, measuring a second respiratory rate with at least one usage independent sensor, comparing the first respiratory rate with the second respiratory rate, and wherein when the first respiratory rate and second respiratory rate are not within a respiratory comparison threshold value, triggering at least one of an alarm or an instruction condition.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates one embodiment of a device for administering oxygen therapy.

FIG. 2 illustrates another embodiment of a device for administering oxygen therapy in communication with a larger hospital information system.

FIG. 3 illustrates a frontal view of a device for administering oxygen therapy.

FIG. 4A-4C depict various example graphical user interfaces (GUIs) that can be displayed on a display of the device in some embodiments.

FIG. 5A-5D depict various example GUIs that can be displayed on a display of the device in some embodiments.

FIG. 6 depicts an example GUI that can be displayed on a display of the device in some embodiments.

FIG. 7 depicts a flowchart of one embodiment of a method for administering gas therapy to a patient.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Disclosed herein are devices and methods for administering gas therapy to a patient. The devices and methods described herein may be administered as gas therapy in a hospital setting, in a home setting, for intermittent use, for chronic use, for treatment of sleep apnea, for treatment of obstructive breathing disorders, for use with non-invasive ventilation devices (e.g., CPAP and BiPAP), for use with mechanical ventilatory devices (e.g., ventilators for hospital, ICU, operative, or home setting), etc.

Disclosed herein are devices and methods for administering oxygen therapy to a patient. By incorporating both user inputs and measured physiological data, the device and related methods are capable of providing a superior adaptive oxygen therapy regimen. This is in part achieved, in various embodiments, by the device's predictive and responsive capacities. As described herein, the components of the device and related system allow for an automated analysis of received inputs and/or measured physiological parameters such that the device can predict an upcoming change in a patient's condition requiring an adjustment in oxygen flow rate and preemptively execute the adjustment as well as respond to any unexpected change in conditions resulting in a needed adjustment in oxygen flow. This demonstrates a marked improvement over current standards of care that require a manual input to affect a change as well as those that use automated systems to merely respond to fluctuations as they occur. Such systems expose patients to the aforementioned risks as it can only make immediate adjustments to the changes as they happen thereby denying healthcare providers the opportunity to intervene earlier if necessary. In at least these ways, the devices and methods described herein provide a significant improvement towards the efficiency and standard of care for patients needing oxygen therapy.

Additionally, the devices as described herein provide an auto-wean function. Patients who no longer need oxygen therapy must be slowly weaned off supplemental oxygen (over a period of minutes to hours depending on their condition) in order to avoid potential health hazards of a premature termination of therapy. Managing such patients to slowly reduce their supply while simultaneously monitoring their oxygen saturation and other parameters to ensure a healthy weaning process can place a substantial administrative burden on hospitals and healthcare providers. In various embodiments, the devices described herein can automate this operation thus maintaining or improving the standard of care for patients coming off oxygen therapy without any increased burden for the hospital or healthcare provider.

Furthermore, the devices as described herein provide improved safety features in the form of a secondary bypass flow path of medical oxygen. Under “normal” operating conditions, the gas can flow through a primary flow path, its flow rate regulated by the various mechanisms described herein. However, when the device detects an error, the system loses power, or by a manual input of a bypass switch, the device redirects the flow of medical oxygen through the bypass flow path. This bypass flow path can comprise flow rate regulator mechanisms that are adjustable and operable by hand without the need for electronic power, thus enabling a reliable delivery of medical oxygen in the advent of some disrupting circumstance.

Further, the devices as described herein may prevent a hypoxemia, or low blood oxygen condition, or reduce a likelihood of developing a hypoxemia status of the patient. As described herein, one or more device components may detect one or more physiological parameters of the patient and adjust oxygen delivery accordingly. The gas delivery, under normal system operating procedures, may be automatic. In other embodiments, gas delivery, through the systems described herein, may be manual or at least partially manual.

As used herein, a “user” may include a patient, a physician, a nurse, a healthcare provider, a caretaker, respiratory therapist, etc. such that the systems, methods, and graphical user interfaces presented by the devices are intuitive and/or easy to use.

### Systems and Devices

The device functions to administer oxygen therapy to a patient. The device is used for medical treatments where the delivery of medical-grade oxygen is advantageous but can additionally, or alternatively, be used for any suitable applications, clinical or otherwise. Although much of the language contained herein is directed to the delivery of medical-grade oxygen gas, the device can be adapted to provide any other gas (e.g., carbon dioxide, molecular nitrogen, volatile anesthetics, etc.) in other embodiments, and thus, the disclosure herein should not be necessarily limited to medical-grade oxygen.

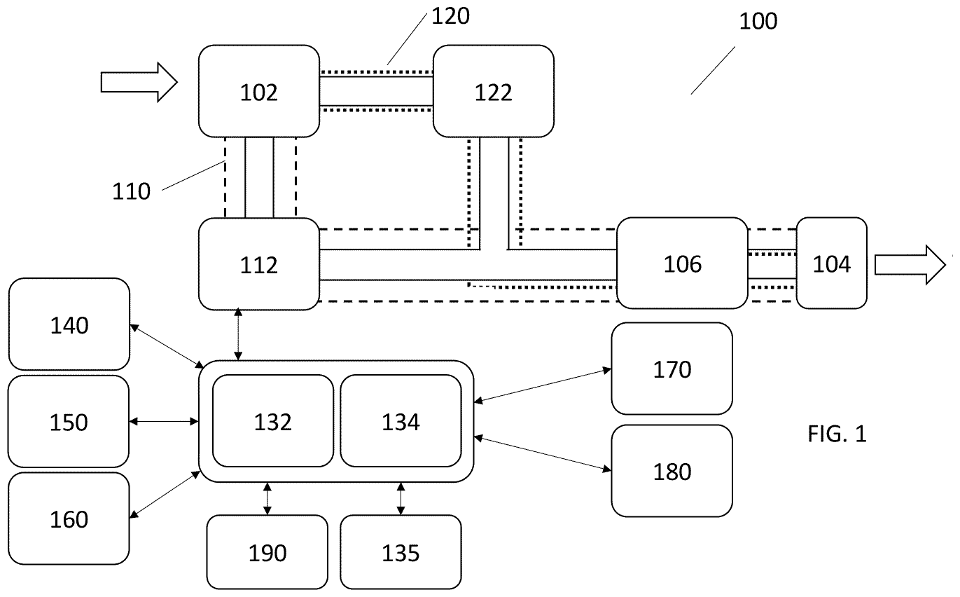

As shown in FIG. 1, one embodiment of a device 100 for delivering gas therapy to a patient comprises a gas intake valve 102 and a gas output connector 104. In many embodiments, the gas intake valve 102 is adapted to receive an incoming supply of medical-grade gas from a gas supply (e.g., a gas cylinder, or a hospital in-house supply system). In some embodiments, a pressure regulator can precede the gas intake valve 102. The gas output connector 104 is configured to attach to various medical devices for the direct delivery of the gas to a patient including, but not limited to, a hose or tube connecting to a venturi mask, non-rebreather facemask, an anesthetic facemask, a Patil-Syracuse facemask, an endoscopy mask, nose cannula, a simple facemask, and a Hudson facemask.

A primary flow path 110 and a bypass flow path 120 fluidly connect the gas intake valve 102 to the gas output connector 104. As described herein, the primary flow path 110 and the bypass flow path 120 can be distinct from each (i.e., sharing no components except for the starting gas intake valve 102 and ending gas output connector 104), or they can share certain lengths of their paths and certain components within (e.g., as is shown in the embodiment of FIG. 1). In some embodiments, the gas intake valve 102 is adapted to block any passage of gas into the system, deliver gas along the primary flow path 110 during a first operating condition, and deliver gas along the bypass flow path 120 during a second operating condition, as described herein. In other embodiments, the gas intake valve 102 is adapted only to direct gas along the primary flow path 110 or the bypass flow path 120, thus requiring another element, such as a flow controller 112, to fully block the passage of gas if desired. In some embodiments, the gas intake valve 102 is a solenoid valve. In some embodiments, the gas intake valve 102 is a three-way solenoid valve. In certain embodiments wherein the primary flow path 110 and the bypass flow path 120 intersect to share a portion of their lengths, a gating valve can be present at their intersection in order to prevent the backfilling of the path not presently in use. In various embodiments, the gating valve can be one or more gate valves, a multiport ball valve, or any other valve appreciated by those of skill in the art.

In many embodiments, the primary flow path 110 comprises a flow controller 112 that is in electronic communication with a processor 132 having a memory 134 storing machine-readable instructions executable by the processor as described herein. Upon receiving a flow rate command from the processor 132, the flow controller 112 articulates its mechanical components to adjust the flow rate of oxygen gas through the first flow path 110. Across various embodiments, the flow controller 112 can employ various mechanical components to create the adjustment, including, but not limited to, a needle valve, a pinch valve, a globe valve, a butterfly valve, a diaphragm valve, or any other control valve appreciated by those of skill in the art.

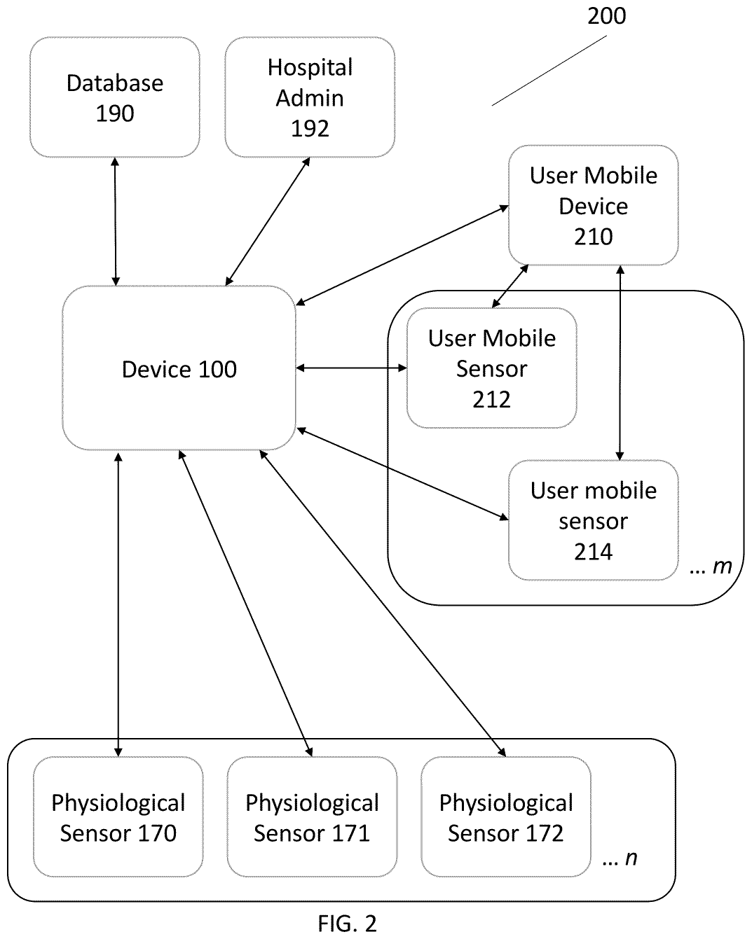

In many embodiments, the bypass flow path 120 comprises a manual flow rate adjuster 122 that is adapted to mechanically control the flow rate through the bypass flow path 120 by a mechanism easily affected by a physical input by a user. As shown in FIG. 2 the manual flow rate adjuster 122 is readily accessible from the exterior of the device 100 to facilitate user input. In some embodiments, the manual flow rate adjuster 122 is a gas regulator. In other embodiments, the manual flow rate adjuster 122 is a standard pressure regulator. In further embodiments, the manual flow rate adjuster 122 is a manually adjustable needle valve.

Returning to FIG. 1, the primary flow path 110 and the bypass flow path 120 each comprise a flowmeter 106, in many embodiments. In some embodiments, each flow path comprises a unique flowmeter 106. In other embodiments, such as the embodiment of FIG. 1, the primary flow path 110 and bypass flow path 120 merge ahead of flowmeter 106 and therefore, share a flowmeter 106. In many embodiments, the flowmeter 106 is a mechanical flowmeter. Embodiments featuring a mechanical flowmeter present the advantage of an additional means of confirming the present flow rate of gas through the device 100 independent of a power supply. In other embodiments, the primary flow path 110 and bypass flow path 120 do not share a flowmeter 106, such that a flow rate through each path is individually confirmable. In some embodiments, the flowmeter 106 is a rotameter or a Thorpe tube flowmeter or another form of analog visualization of flow rate. In other embodiments, the flowmeter 106 is a digital flowmeter that can similarly be used to confirm an actual flow rate in one or both of the primary flow path 110 and bypass flow path 120. In some embodiments wherein the primary flow path 110 and bypass flow path 120 each comprise a unique flowmeter 106, the flowmeter 106 of one of the flow paths can be a mechanical flowmeter while that of the other flow path can be a digital flowmeter. In further embodiments, one or both of the primary flow path 110 and the bypass flow path 120 can comprise a unique flowmeter 106 in addition to a shared flowmeter 106. For example, an embodiment of the device 100 can comprise a primary flow path 110 with a unique flowmeter and a shared flowmeter. In this example, the unique flowmeter of the primary flow path 110 could only measure the flow rate through the primary flow path 110, while the shared flowmeter could be positioned downstream of a merging point between the primary flow path 110 and bypass flow path 120 in order to be capable of measuring the flow rate through both the flow paths 110 and 120. Across various embodiments, the one or more flowmeters 106 can be adapted to verify an actual flow rate in one or both of the flow paths 110 and 120; to provide reference flow rate data during a calibration of the device 100; and to intermittently sample the flow rate through the bypass flow path 120.

In addition to being in communication with the flow controller 112, the processor 132 can be in communication with a bypass switch 135, at least one physiological sensor 170, a display 140, a user input/output device 150, and/or an alarm 160, in various embodiments. In further embodiments, the processor 132 can be in communication with at least one internal sensor 180 and at least one external electronic patient records database 190. The device 100 can also be connected to a power supply (not shown) and can further comprise a power reserve (not shown) such as a rechargeable battery that allows the continued operation of the device 100 in case of loss of power from the power supply.

In many embodiments, the device 100 further comprises a bypass switch 135. Under a first operating condition (a “normal” operating condition) medical gas flows through the primary flow path 110. Under a second operating condition, the gas intake valve 102 instead directs medical gas through the bypass flow path 120. Furthermore, the device 100 is configured to change from the first operating condition to the second operating condition when a bypass criterion is detected or inputted. In many embodiments, a bypass criterion can include, but is not limited to, a loss of device power, a system error, or a user selection of the emergency bypass switch 135. The recognition of a bypass criterion can, in various embodiments, be implemented by mechanical hardware or software systems for various bypass criteria independently. For example, in the advent of a loss of system power, the hardware of the device 100 can be adapted to automatically toggle a state of the gas intake valve 102 (e.g., a solenoid valve) such that the device 100 enters its second operating condition, directing gas down the bypass flow path 120. In a further example, software implemented on the device 100 can detect a system error bypass criterion if it determines a discrepancy or malfunction among various components (e.g., the flow controller 112, the flowmeter 106, one or more physiological sensors 170, etc.) in various embodiments. In still another example, a user selection of the emergency bypass switch 135 can be performed digitally or mechanically in various embodiments. In some embodiments, the emergency bypass switch 135 can be a physical switch that when toggled initiates the hardware of the device to enter the second operating condition. In other embodiments, the emergency bypass switch 135 can be an interactable element displayed to a user on a graphical user interface on a display 140 of the device 100. In these embodiments, when the digital emergency bypass switch 135 is toggled, software systems initiate the change into the second operating condition. In some embodiments, the device 100 can include both a physical and digital emergency bypass switch 135. In many embodiments, the device 100 will return to the first operating condition according to an appropriate user input. In some embodiments, this user input can be a toggling of the emergency bypass switch 135.

As described herein, a variety of physiological sensors 170 can be employed by the device 100, allowing the device 100 to measure physiological parameters of the patient receiving gas therapy. In many embodiments, these measured physiological parameters affect the automatic adjustments the device 100 makes to provide improved gas therapy, as described herein. Example physiological sensors 170 can include, but are not limited to, an SpO2 sensor, an EtCO2 sensor, an accelerometer, a microphone, a heart rate monitor, a back pressure monitor, an SpO2 waveform monitor, an EKG, or a combination thereof. In various embodiments, the measured physiological parameters acquired by one or more of the above sensors can include, but are not limited to, an oxygen saturation level, a rate of change of an oxygen saturation level, an EtCO2 level, a rate of change of an EtCO2 level, a respiratory rate, a rate of change of a respiratory rate, a heart rate, a rate of change of a heart rate, a motion of the patient, an activity level of the patient, an audio signal of a patient breathing, a cyclic back pressure, a chest rise monitoring, or a combination thereof.

In some embodiments, a patient's respiratory rate can be monitored by a plurality of physiological sensors 170 independently. Some physiological sensors 170 can measure a respiratory rate that inherently depends upon the patient's inhalation of medical oxygen from the device 100 (i.e., a “usage dependent” sensor). Examples of usage dependent physiological sensors 170 can include, but are not limited to, an EtCO2 monitor recording capnography data, and a pressure sensor performing cyclic back pressure monitoring within a tube connecting the gas output connector 104 to the patient. Other physiological sensors 170 can measure a respiratory rate that does not depend upon the patient's inhalation of medical oxygen from the device (i.e., a “usage independent” sensor). Examples of usage independent physiological sensors 170 can include, but are not limited to, a pulse oximeter, a microphone detecting an audio signal of a patient's breathing, an accelerometer measuring the movement of the patient's chest wall, an an oxygen sensor, a carbon dioxide sensor, one or more electrodes measuring electrocardiogram signals, and/or any combination thereof. In some embodiments, the device 100 can compare a respiratory rate measured by one or more usage dependent sensors with that of one or more usage independent sensors. If a discrepancy between the two respiratory rates is detected, the device 100 may trigger an alarm condition, an instruction condition, or a bypass criterion as described herein.

A discrepancy between a first respiratory rate measured by a usage dependent sensor and a secondary respiratory rate measured by a usage independent sensor can be determined by comparing the first and second respiratory rates to see if they are within a respiratory comparison threshold value. In certain embodiments, the respiratory comparison threshold value is about 5% to about 0.5% of one of the first or second respiratory rates. In other embodiments, the respiratory comparison threshold value is about 2.5% to about 0.5% of one of the first or second respiratory rates. In further embodiments, the respiratory comparison threshold value is about 1% to about 0.5% of one of the first or second respiratory rates. In some embodiments, the respiratory comparison threshold value is about 5% of one of the first or second respiratory rates. In other embodiments, the respiratory comparison threshold value is about 2.5% of one of the first or second respiratory rates. In further embodiments, the respiratory comparison threshold value is about 1% of one of the first or second respiratory rates. In still further embodiments, the respiratory comparison threshold value is about 0.5% of one of the first or second respiratory rates. In additional embodiments, the respiratory comparison threshold value is less than about 0.5% of one of the first or second respiratory rates.

In many embodiments, the detection of a respiratory rate discrepancy can indicate an “improper” use of the device 100 (i.e., that a user of the device 100 has inadequately equipped and/or implemented the various features of the device 100 as described herein, thereby preventing the device 100 from detecting and/or affecting a change in the patient's SpO2 to the degree that the device 100 is capable.)

In some embodiments, the device 100 comprises a display 140 and a user input/output device 150. The display 140 can allow the device 100 to present information to a user (e.g., healthcare provider, patient, etc.) about the operation of the device 100 and its settings (e.g., current flow rate, etc.) The input/output device 150 can allow a user to input information and make selections or decisions on the device (i.e., user input). In certain embodiments, user input can include, but is not limited to, one or more of a target oxygen saturation level or a minimum oxygen saturation level. In some embodiments, such as the embodiment of FIG. 2, the input/output device 150 is merged with the display 140 in the form of a touchscreen. In other embodiments, the input/output device 150 can be physical buttons or dials on a surface of the device 100 accessible by a user. In still other embodiments, the device 100 can be adapted to communicate with a portable electronic device (not shown), such as a cell phone or tablet, carried by a user. One of skill in the art will appreciate the wide variety of network protocols that can be employed to connect a portable electronic device to the device 100, including but not limited to Bluetooth, Wi-Fi, and a cellular network. In these embodiments, the portable electronic device can be used to submit user input to the device. In some of these embodiments, the device 100 can forgo a display of its own, communicating all relevant information to a display on the portable electronic device.

In some embodiments, the input/output device 150 can comprise a speaker. In some embodiments, the speaker can play prerecorded voice messages to a user when the device detects an instruction condition. Instruction conditions can include, but are not limited to, a detection of a misplaced physiological sensor 170, a detection of a respiratory rate discrepancy (as described herein), and a device calibration. For example, if the processor 132 of the device 100 detects that a physiological sensor 170 is recording improper or null data, the device can consider this a misplaced physiological sensor instruction condition. In this embodiment, it can then play a prerecorded audio message instructing a user to readjust, fix, or confirm proper placement of the physiological sensor 170. As another example, if the processor 132 detects a respiratory rate discrepancy instruction condition, the device can play a prerecorded audio message instructing a user to adjust one or more settings (e.g., provide a new target oxygen saturation level) or to readjust, fix, or confirm proper placement of one or more physiological sensors. Additionally, or alternatively, a display 140 of the device 100 may provide a prompt, pop-up, or other message or alert that includes the instruction condition and one or more remedies or solutions for the instruction condition. In additional embodiments, when a physiological sensor 170 is attached to a patient or at any time during operation of the device 100, the device 100 can employ the speaker to play a prerecorded audio message instructing a user to perform a certain task (e.g., a movement of a body part, an intentional change in respiratory rate, etc.) in order to calibrate the physiological sensor 170. In these embodiments, this operation can be considered a device calibration instruction condition.

In some embodiments, the device 100 further comprises an alarm 160. Across various embodiments, the alarm 160 can be one or all of an auditory, visual, or message-based alarm. Examples of auditory alarms can include, but are not limited to, a ringing, a beeping, a buzzing, etc. In some embodiments, a speaker of the input/output device 150 can be employed to project the auditory alarm. Examples of visual alarms can include, but are not limited to, a colored light or a blinking light. Exemplary message-based alarms are configured to display a message on a display 140 of the device and/or to send an electronic message to another device (e.g., user's mobile phone, another computer system such as that of a hospital, etc.) Message-based alarms can allow for the discreet notification of relevant parties, including those not in proximity to the physical device 100. In some embodiments, the message-based alarms can trigger secondary visual or auditory alarms (not shown) not physically attached to the device 100 (e.g., such as an alarm located at a nurses' station in a hospital). The device 100 can trigger the alarm 160 for a variety of purposes in various embodiments. In one embodiment, the device 100 can trigger the alarm 160 when a measured oxygen saturation level (acquired by a physiological sensor 170) falls below a received minimum oxygen saturation level (acquired by user input). In another embodiment, the device 100 can trigger the alarm 160 when a respiratory rate discrepancy is detected. In some embodiments, the device 100 can have a plurality of alarms that are triggered for unique circumstances, herein referred to as alarm conditions.

In many embodiments, a plurality of alarm conditions can occur simultaneously. In some embodiments, a unique alarm 160 can trigger to indicate the specific combination of alarm conditions. For example, a unique colored light can be used as a visual alarm to indicate a certain set of alarm conditions. In other embodiments, a mixture of alarm types (e.g., visual, auditory, and message-based) can be employed. For example, all alarm conditions can trigger a singular auditory alarm, but a unique message-based alarm can be displayed on a display 140 of the device 100 to indicate the one or more triggering alarm conditions. Various combinations of alarm types are considered herein, and a broad variety can be employed without deviating from the scope of this disclosure.

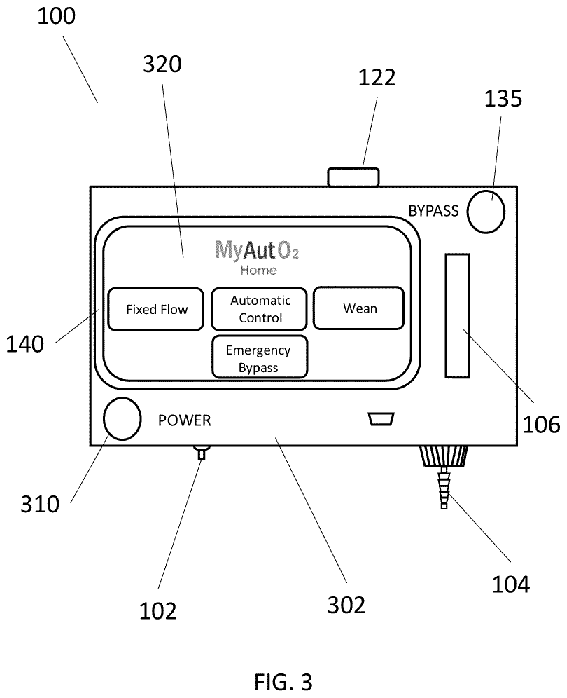

In some embodiments, the device 100 further comprises at least one internal sensor 180. In many embodiments the at least one internal sensor 180 is located beneath a housing 302 (see FIG. 3) of the device 100 to more accurately detect various conditions of the components of the device 100. In certain embodiments, one or more of the at least one internal sensor 180 can be integrated into one or both of the primary flow path 110 and bypass flow path 120 to detect conditions or qualities of the medical oxygen flowing through the device 100. In some embodiments, the at least one internal sensor 180 can include, but is not limited to, a humidity sensor or a pressure sensor. For example, a humidity sensor can detect when a humidity inside the device 100 exceeds a predetermined baseline tolerance and, in some embodiments, can trigger the alarm 160 (via a humidity alarm condition) and/or establish a bypass criterion of a system error. In certain embodiments, an increased internal humidity can be indicative of a malfunction of the device 100. In another example, a pressure sensor can detect when an air pressure level inside the device 100 exceeds a predetermined baseline tolerance, and in some embodiments, can trigger the alarm 160 (via a pressure alarm condition) and/or establish a bypass criterion of a system error. In certain embodiments, an increased internal pressure can be indicative of a leak in or malfunction of one or both of the primary flow path 110 and the bypass flow path 120. In another embodiment, the pressure sensor can detect gas pressure inside one or both of the primary and bypass flow paths and can trigger the alarm 160 and/or establish a bypass criterion of a system error when the gas pressure drops beneath a predetermined baseline, indicating either a leak or malfunction in the device 100 or a depletion of the incoming gas supply.

In some embodiments, the device 100 can be in communication with at least one external electronic patient records database 190. The patient records database 190 can be stored on an external computer or computing system (e.g., a cloud computing system), and the database 190 can store various medical information regarding a user or patient of the device 100. In some embodiments, the device 100 can be adapted to retrieve relevant user information for use in administering automated gas therapy, as described herein. This user information can include, but is not limited to, biographic/demographic information, past and/or present diagnoses, past and/or present medication, medical test scores, etc. In further embodiments, the device 100 can be adapted to write information to the database 190. Various information can be written to the database 190 across various embodiments. In some embodiments, the device 100 writes at least a subset of the measured physiological parameters recorded by the at least one physiological sensor 170. In some embodiments, the device 100 writes an event history comprising a timestamped log of any triggering of the alarm 160, bypass criterion, or changes in user input. In some embodiments, the device 100 writes a timestamped log of a predicted oxygen saturation level (as described herein, see below.) In some embodiments, the database 190 is an electronic health records database. In other embodiments, the database 190 is an electronic medical record database. In further embodiments, the database 190 is a personal health record database. For fast identification and retrieval of relevant patient information, the device 100 can further comprise an optical scanner (not shown) such as a barcode or QR-code scanner capable of reading an appropriate code on a patient's person (e.g., a wristband provided by the hospital) that identifies the patient's records in the database 190.

FIG. 2 depicts device 100 for administering gas therapy integrated into and in communication with a larger hospital information system 200. In many embodiments, the device 100 can be in communication with a plurality of physiological sensors 170, 171, and 172, as well as with a user mobile device 210. In some embodiments, the user mobile device 210 (e.g., a tablet, a cellular device, a smartwatch, etc.) can be used to submit user inputs (e.g., a target oxygen saturation level, etc.) to the device 100. In further embodiments the device 100 and the user mobile device 210 can be in communication with one or more user mobile sensors 212 and 214. In many embodiments, these user mobile sensors 212 and 214 are third-party physiological sensors worn by the patient, sometimes integrated into the user mobile device 210 (e.g., electrodes on a patient's smartwatch). In other embodiments, the user mobile sensors 212 and 214 are physically distinct devices from the user mobile device 210. In various embodiments, these user mobile sensors 212 and 214 can include, but are not limited to, an SpO2 sensor, an EtCO2 sensor, an accelerometer, a microphone, a heart rate monitor, a back pressure monitor, an SpO2 waveform monitor, an EKG, or a combination thereof. The device 100 can communicate with these one or more user mobile sensors 212 and 214, either directly or via the user mobile device 210, to expand the total number of physiological sensors available.

In various embodiments, the device 100 is also in communication with a database 190 of electronic patient records as described herein. Furthermore, in embodiments wherein the device 100 is present in hospital, the device 100 can be in communication with a hospital administration network 192. In various embodiments, the hospital administration network 192 comprises one or more computers maintaining logistical records of patient locations and statuses. By being in communication with a hospital administration network 192, the device 100 can present the measured physiological parameters to healthcare providers (e.g., doctors, nurses, etc.) as well as receive user inputs (e.g., a target oxygen saturation level) from healthcare providers without the healthcare provider being physically present at the device 100. A variety of network protocols can be employed to maintain communication between the device 100 and the physiological sensors 170, 171, and 172, the user mobile device 210 and its one or more user mobile sensors 212 and 214, the database 190 and the hospital administration network 192 including, but not limited to, a wired internet protocol, a wireless internet protocol (Wi-Fi), a cellular network, Bluetooth, and any other communication protocol appreciated by those of skill in the art.

FIG. 3 depicts an exterior view of one embodiment of the device 100 for administering gas therapy to a patient. In this figure, many of the internal components of the primary flow path 110 and bypass flow path 120 are obscured by a housing 302, however, the gas intake valve 102, gas output connector 104, and flowmeter 106 are visible in this embodiment. The manual flow rate adjustor 122 is also visible and is sufficiently exterior to the housing 302 to allow a user to easily adjust its physical controls. The device 100 also presents an optional display 140 showing an exemplary graphical user interface (GUI) 320. In the embodiment of FIG. 2, the display 140 and input/output device 150 are integrated as one element in the form of a touchscreen. The device 100 can also comprise an accessible power switch 310 and bypass switch 135. In some embodiments, the device 100 does not include display 140, such that the display is on a portable device or other computing device. In such embodiments, data from device 100 is transmitted, wirelessly or via a wired connection, to the portable device or computing device.

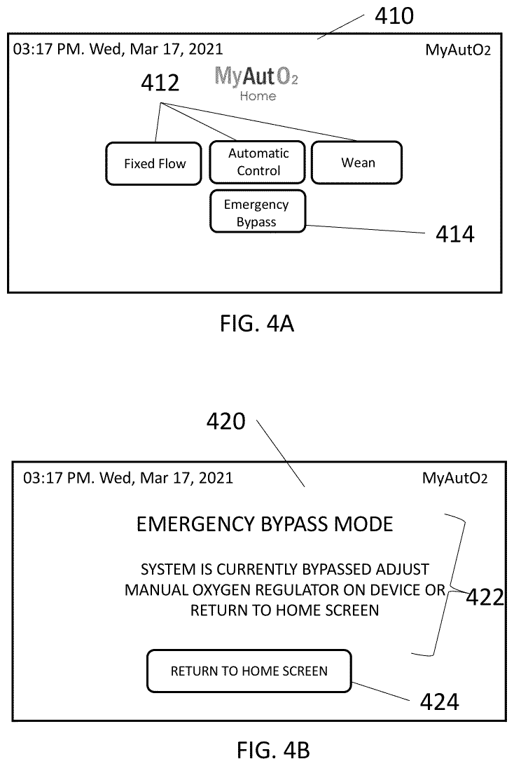

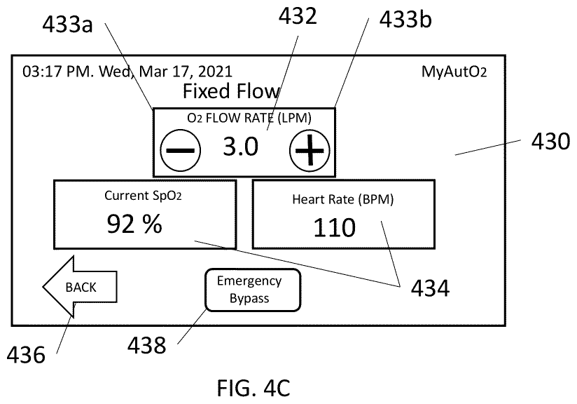

FIGS. 4A-4C, 5A-5D, and 6 depict various exemplary graphical user interfaces (GUIs) that can be displayed on the display 140 of the device or a portable device, in various embodiments. In the embodiments of FIGS. 4A-4C, 5A-5D, and 6, the GUIs are adapted to be displayed on a touch screen display. In other embodiments lacking a touchscreen, the GUIs can be modified or adapted to be more convenient for other means of receiving user input. In some embodiments, additional example GUIs can include those for the inputting of patient information.

FIG. 4A depicts an exemplary “home screen” 410 for the device 100 presenting a series of mode buttons 412, selectable by a user, that enable the device 100 to operate in various modes. In some embodiments, the mode buttons 412 can include a “fixed flow” mode, an “automatic control” mode, and a “wean” mode as described herein. In alternate embodiments, other modes can be displayed. The home screen 410 can also include an emergency bypass button element 414 that can input a bypass criterion as described herein. Many of the GUIs across various embodiments can include an emergency bypass button element 414 to provide convenient access to that functionality to users at all times.

FIG. 4B depicts an example GUI 420 that is displayed when the device 100 is operating under the second operating condition (i.e., while the medical gas is being delivered through the bypass flow path 120). The GUI 420 can provide a clear message 422 indicating that the device 100 is operating under the second operating condition. One or more navigation buttons 424 can be present to return the user to other GUIs, such as the home screen GUI example of FIG. 4A. In some embodiments, selecting the one or more navigation buttons 424 can reset the device 100 to the first operating condition. In other embodiments, a user can be required to toggle an emergency bypass button (not shown), such as a mechanical emergency bypass switch in order to return the device 100 to the first operating condition.

FIG. 4C shows an example GUI 430 that is displayed when the device is in a “fixed flow mode.” As used herein, when the device 100 is in “fixed flow mode,” the device 100 simply administers a flow of medical gas to the patient at a static flow rate determined by user input. In many embodiments, the first and second operating conditions as described herein can apply during fixed flow mode. An example GUI 430 for fixed flow mode can comprise a display of the current flow rate 432 as well as selectable elements 433a and 433b for increasing or decreasing the flow rate. In various embodiments, the GUI 430 can also include current measured physiological parameters 434. In the embodiment of FIG. 4C, the GUI 430 includes a measured SpO2 as well as a measured heart rate, but other physiological parameters can be displayed in alternative embodiments. The GUI 430 can also include at least one navigational button 436 that, when selected, directs the user to another GUI, such as the home screen GUI of FIG. 4A. In some embodiments, the device 100 terminates fixed flow mode when a navigational button 436 is selected. In other embodiments, the device 100 will maintain fixed flow mode until another mode of operation is initiated. In many embodiments, the GUI 430 includes an emergency bypass button element 438 that can input a bypass criterion to the device 100 as described herein. In many embodiments, a selection of the emergency bypass button element 438 will direct the user to another GUI, such as the GUI of FIG. 4B.

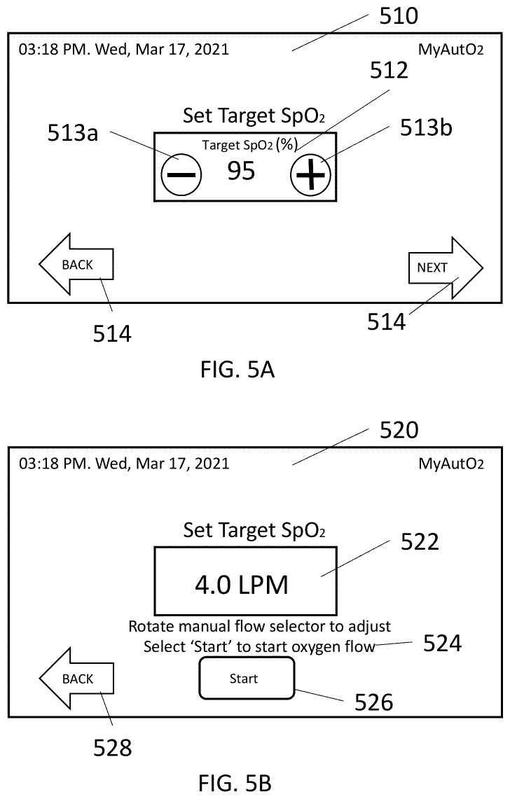

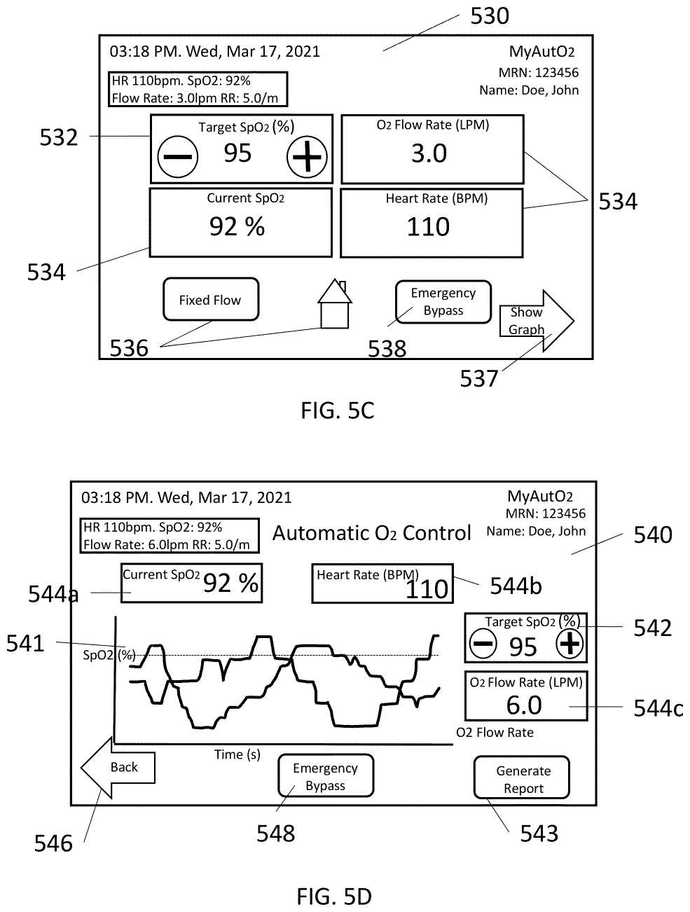

FIGS. 5A-5D depict a series of example GUIs that are displayed when operating in “automatic control” mode. In various embodiments, the exemplary GUIs can be displayed in various subsets or orders without deviating from the scope of this disclosure. In some embodiments, upon the selection of automatic control mode (e.g., such as on home screen GUI of FIG. 4A), a user will be prompted for a target and/or minimum oxygen saturation level. In FIG. 5A, the example GUI 510 requests a user input for a target oxygen saturation level. A target oxygen saturation level value 512 can be displayed in addition to selectable elements 513a and 513b for increasing or decreasing the target oxygen saturation level value 512. At least one navigation button 514 can be present to direct the user to other GUIs when selected. In other embodiments, a GUI similar to that of FIG. 5A can be used for the inputting of a minimum oxygen saturation level

In FIG. 5B, the example GUI 520 requests a user to confirm the backup flow rate (i.e., the flow rate determined by the manual flow rate adjuster during the secondary operating condition). The backup flow rate value 522 can be presented on the GUI 520, along with a message 524 instructing the user to confirm the value on the manual flow rate adjuster. In some embodiments, additional or alternate messages 524 can be displayed. A selectable start button 526 can, in many embodiments, initiate the automatic flow of medical gas to the patient as described herein. In many embodiments, a selection of the start button 526 will direct a user to an additional GUI, such as the automatic control mode GUIs of FIG. 5C or 5D. At least one navigation button 528 can direct the user to other GUIs.

FIG. 5C shows one example of a GUI 530 that is displayed during the operation of an automatic control mode as described herein. The GUI 530 can comprise a target SpO2 value 532 with or without selectable elements for increasing or decreasing the target SpO2 value 532, one or more measured values 534, an emergency bypass button element 538, and at least one navigation button 536. In the embodiment of FIG. 5C, the displayed measured values 534 include a current gas flow rate, and the measured physiological parameters of an oxygen saturation level and a heart rate. In alternate embodiments, other measured values 534 can be displayed. At least one navigation button 536 can direct the user to other GUIs. In some embodiments, the GUI 530 can comprise a “display graph” button 537 that can direct a user to another GUI, such as that of FIG. 5D, that displays a chart of graph of certain values.

FIG. 5D shows one example of a GUI 540 that is displayed during the operation of an automatic control mode as described herein. Similar to the example of FIG. 5C, the GUI 540 can comprise a displayed target oxygen saturation level 542, displays of measured values 544, an emergency bypass button element 548, and at least one navigation button 546. In many embodiments, the GUI 540 further comprises a graph 541 that displays various values (e.g., a measured oxygen saturation level, a flow rate, a minimum oxygen saturation level, etc.). In many embodiments, these values are plotted over time. In some embodiments, the GUI 540 further comprises a “generate report” button 543, that instructs the device 100 to record and/or transmit a therapeutic summary comprising at least a subset of the values to a patient medical records database (such as the database 190 of FIG. 1). In some embodiments, the therapeutic summary comprises a risk stratification analysis.

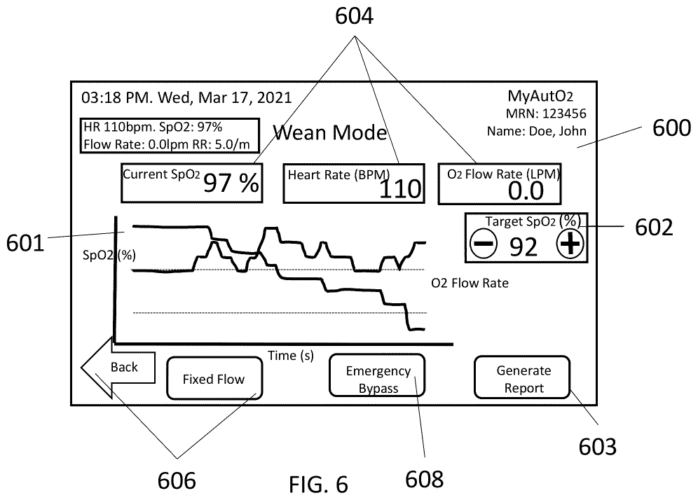

FIG. 6 depicts an example GUI 600 displayed during a “wean” mode of the device. As described herein, a “wean” mode or “auto-wean” of the device 100 allows for a slow reduction of at least one of the target oxygen saturation level or flow rate to introduce an unassisted physiological level. During this operation, an example GUI 600 can comprise a minimum oxygen saturation level 602 with or without selectable elements for increasing or decreasing the minimum oxygen saturation level, one or more displayed values 604, at least one navigation button 606, a graph 601, and an emergency bypass button element 608. In some embodiments, the at least one navigation button 606 can include a button to switch the device to a fixed flow mode as described herein. In some embodiments, the GUI 600 can include a “generate report” button 603 that instructs the device 100 to record and/or transmit a therapeutic summary comprising at least a subset of the values to a patient medical records database (such as the database 190 of FIG. 1). In some embodiments, the therapeutic summary comprises a risk stratification analysis.

### Methods

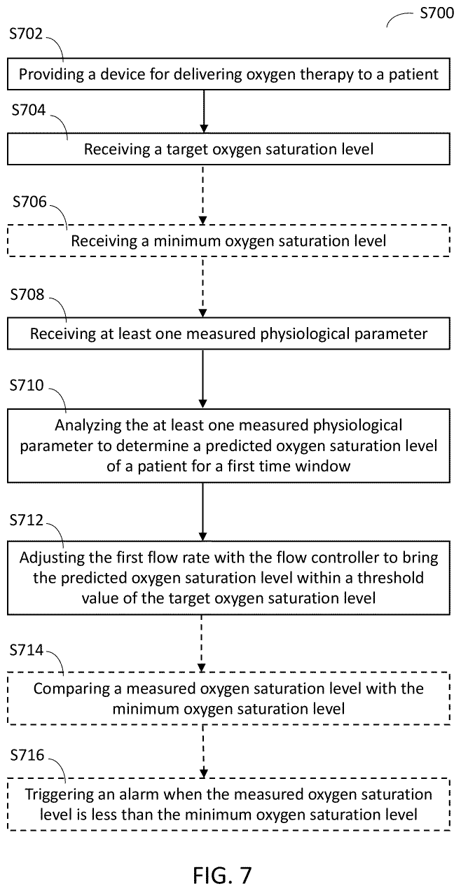

As shown in FIG. 7, a method 700 for delivering oxygen therapy to a patient of one embodiment includes providing a device for delivering oxygen therapy in block S702, receiving a target oxygen saturation level in block S704, optionally receiving a minimum oxygen saturation level in optional block S706, receiving at least one measured physiological parameter in block S708, analyzing the at least one measured physiological parameter to determine a predicted oxygen saturation level of a patient for a first time window in block S710, adjusting a first flow rate with a flow controller to bring the predicted oxygen saturation level within a threshold value of the target oxygen saturation level S712, optionally comparing a measured oxygen saturation level with the minimum oxygen saturation level in optional block S714, and optionally triggering an alarm when the measured oxygen saturation level is less than the minimum oxygen saturation level in block S716. The method functions to deliver gas therapy to a patient. In some embodiments, the method functions to automatically deliver adaptive gas therapy to a patient according to the patient's needs.

At block S702, the method 700 includes providing a device for delivering oxygen therapy to a patient. In many embodiments, the device comprises a gas intake valve, a primary flow path connecting the gas intake valve to a gas output connector, the primary flow path comprising a flow controller configured to adjust a first flow rate through the primary flow path, and at least one physiological sensor. In various embodiments, the at least one physiological sensor can include, but is not limited to, an SpO2 sensor, an EtCO2 sensor, an accelerometer, a microphone, a heart rate monitor, a back pressure monitor, an SpO2 waveform monitor, and an EKG. In some embodiments, the device further comprises an alarm. In further embodiments, the device is the device of FIGS. 1-3.

At block S704, the method 700 includes receiving a target oxygen saturation level. The target oxygen saturation level can be a desired oxygen saturation level at which the method 700 is to maintain in a patient by administering gas therapy. In many embodiments, the target oxygen saturation level is received at the device. In some embodiments, the target oxygen saturation level is received by user inputs via an input/output device facilitated by a display. In some embodiments, the user input specifies an exact target oxygen saturation level desired by the patient. In other embodiments, the user input represents various biographical, demographical, and/or medical information regarding the patient from which the target oxygen saturation level is calculated. In still further embodiments, the relevant user biographical, demographical and/or medical information is retrieved from a database of patient medical records as described herein.

At optional block S706, the method 700 optionally includes receiving a minimum oxygen saturation level. The minimum oxygen saturation level can be a lowest oxygen saturation level at which the method 700 is allowed to maintain in a patient by administering gas therapy. In many embodiments, the minimum oxygen saturation level is received at the device. In some embodiments, the minimum oxygen saturation level is received by user inputs via an input/output device facilitated by a display.

At block S708, the method 700 includes for receiving at least one measured physiological parameter. In various embodiments, the at least one measured physiological parameter can include, but is not limited to, an oxygen saturation level, a rate of change of an oxygen saturation level, an EtCO2 level, a rate of change of an EtCO2 level, a respiratory rate, a rate of change of a respiratory rate, a heart rate, a rate of change of a heart rate, a motion of the patient, an activity level of the patient, an audio signal of a patient breathing, a cyclic back pressure, a chest rise monitoring, or a combination thereof. In many embodiments, the at least one measured physiological parameter is collected by the at least one physiological sensor of the device.

Data representing the at least one measured physiological parameter can be received at a variety of speeds in different embodiments. In some embodiments, the data representing the at least one measured physiological parameter can be received about once every minute to about once every half a second. In other embodiments, the data representing the at least one measured physiological parameter can be received about once every thirty seconds to about once every half a second. In further embodiments, the data representing the at least one measured physiological parameter can be received about once every five seconds to about once every half a second. In still further embodiments, the data representing the at least one measured physiological parameter can be received about once every second to about once every half a second. In additional embodiments, the data representing the at least one measured physiological parameter can be received about once every minute, about once every thirty seconds, about once every five seconds, about once every second, about once every half a second, and as fast as the physiological sensor is capable of recording and recording, which for many sensors, is equivalent to “live” updates, which occur multiple times a second. In further embodiments receiving more than one measured physiological parameter, the data representing each physiological parameter can be received at the same or different rates.

At block S710, the method 700 includes for analyzing the at least one measured physiological parameter to determine a predicted oxygen saturation level of a patient for a first time window. As the patient's condition changes (as represented in the received data and signals representing the measured physiological parameters), the patient's upcoming need for medicinal oxygen can change accordingly, and the current flow rate may be either insufficient or excessive. In some circumstances, increased physical activity as represented by an activity level recorded by an accelerometer, for example, can indicate an upcoming increased need for medical oxygen as the patient more rapidly consumes the oxygen presently stored in his or her blood. Conversely, in some embodiments, subtle changes in a patient's respiratory rate can indicate a new baseline oxygen consumption at which a current flow rate of medical oxygen is unnecessarily abundant and therefore wasteful.

In some embodiments, analyzing comprises extracting one or more features from the received sensor signal(s) of the measured physiological parameters. For example, the one or more features may include an amplitude, a complexity of the signal, a periodicity of the signal, a rate of onset, a rate of decay, a slope, etc. The one or more features may be used to determine or calculate a predicted oxygen saturation level of the patient at or during the first time window. In some embodiments, analyzing may further include artifact removal, for example removing patient movement artifacts from a respiratory signal or rate. In some embodiments, analyzing may include generating a baseline physiological parameter value for a given measured physiological parameter and comparing the at least one measure physiological parameter at a later time to the baseline physiological parameter to determine, for example a rate of change. In other embodiments, analyzing can include correlating a plurality of measured physiological parameters to determine or calculate a predicted oxygen saturation level of the patient at or during the first time window.

In many embodiments, the first time window is the projected time point in the future at which the method 700 considers a predicted oxygen saturation level. In some embodiments, the first time window is about 1 second to about 2.5 hours; about 1 second to about 1 hour; about 1 minute to about 1 hour; about 1 minute to about 10 minutes; 1 minute to about 5 minutes; about 5 minutes to about 1 hour; is about 5 minutes to about 30 minutes. In some embodiments, multiple projected time points are considered and maintained simultaneously.

In some embodiments, analyzing the at least one measured physiological parameter to determine a predicted oxygen saturation level of a patient for a first time window may include analyzing capillary oxygen saturation of the patient. For example, oxygen saturation data may be received from an oxygen sensor (e.g., a pulse oximeter) capturing data before and/or during the first time window. In some embodiments, the oxygen data may be stored for future comparisons, calculations, and/or analysis. In some embodiments, the stored oxygen data may be provided as input to the analysis that is associated with the data corresponding to measurements taken during a previous time window (e.g., the first time window).

In some embodiments, the measured physiological data may be the capillary oxygen saturation data. Such data may include patient-specific historical values (and/or historical trends) associated with the measured physiological parameter (e.g., measured capillary oxygen saturation data). The patient-specific historical values may include or otherwise correspond to a historical oxygen saturation range, a historical oxygen saturation volatility, a historical oxygen saturation responsiveness, a volume of oxygen supplementation historically required to achieve and/or maintain a target oxygen saturation level, a change over time in the volume of oxygen supplementation historically required to achieve and/or maintain a target oxygen saturation level, a volume of oxygen supplementation historically required to achieve and/or maintain a target oxygen saturation level proportionally to one or more vital signs (e.g., respiratory rate, heart rate, etc.), and/or any combination thereof. In some embodiments, the analysis may further include performing analyses to assess a previous oxygen therapy. A waveform analysis may be performed using data collected to determine whether the data is of a quality above a predefined data quality threshold.

In some embodiments, the analysis may further include analyzing motion (e.g., activity) of the patient using an accelerometer sensor or other activity monitor that measures and categorizes motion over time and stores such motion as activity data. Such activity data may be received by the device administering oxygen therapy. The activity data may include continuously tracked activity data or windows of tracked activity data corresponding to patient activity levels. The analysis may include correlating the received activity data with subsequent oxygen requirement changes. The correlation may be used to determine a relationship between treatment of the patient and response of the patient. The correlation and/or relationship determination may be used to begin, adjust, or end oxygen treatment for the patient.

In some embodiments, the measured physiological data may be electrocardiography data. The analysis of the physiological data may include assessing electrocardiography data associated with the patient. For example, one or more electrode sensors may be placed on the patient to measure electrocardiogram (ECG) signals to monitor, track, and/or store heart rhythm/activity signals. Alternatively, electrocardiography data may be fed to the device from an existing data capture source. The analysis may include determining a correlation and/or relationship between changes in ECG signals (e.g., heart rate) and subsequent metric changes including capillary oxygen saturation and oxygen therapy requirements over time. In some embodiments, the analysis may include determining a correlation and/or relationship between an amount of supplemental oxygen provided and heart rate responsiveness to the supplemental oxygen. The correlation and/or relationship determination may be used to begin, adjust, or end oxygen treatment for the patient.

In some embodiments, the measured physiological data may be end-tidal carbon dioxide (ETCO2) monitoring data (e.g., capnometry data). For example, the patient may be receiving oxygen through a tube. Within or connected to the tube, a carbon dioxide sensor may be installed to assess gas samples aspirated from exhaled gas flow from the patient. The gas samples may be analyzed to measure carbon dioxide production and clearance (e.g., ventilation). The analysis may include determining a correlation and/or relationship between the gas samples and a level of oxygen provided to the patient. The correlation and/or relationship determination may be used to begin, adjust, or end oxygen treatment for the patient.

In a non-limiting example, capillary saturation data may be received by the at least one physiological sensor (e.g., a pulse oximeter, a microphone detecting an audio signal of a patient's breathing, and an accelerometer measuring the movement of the patient's chest wall, an an oxygen sensor, a carbon dioxide sensor, one or more electrodes measuring electrocardiogram signals, and/or any combination thereof). The data may be verified for quality using one or more predefined quality thresholds. Upon determining that at least a portion of the data exceeds the one or more predefined quality thresholds, the portion of the data that exceeds the one or more quality thresholds may be compared to a predefined target data set. The target data set may pertain to a target oxygenation regimen, a target oxygen saturation level, a target heart rate, a target carbon dioxide level, a target respiratory rate, or any combination thereof. The analysis may further include verifying a respiratory rate to ensure an oxygen therapy is being delivered and administered to the patient without equipment error, operator error, and the like. In some embodiments, ancillary data (e.g., heart rate, end-tidal CO2, activity monitor, respiratory rate, etc.) may also be evaluated to determine a likelihood that a current capillary saturation point will deviate from a current value in the absence of therapeutic changes based on a particular patient's historical values and/or historical trends. The analysis may further include determining a predicted oxygen saturation value and comparing the predicted oxygen saturation value to a target oxygen saturation value. In addition, a current oxygen flow rate may be measured and/or verified. In response to determining that the patient's current oxygen flow rate is not within a predefined threshold amount of the target oxygen flow rate, a new (e.g., updated) target oxygen flow rate may be determined based on the predicted oxygen saturation value (e.g., positive or negative value) and the current oxygen flow rate. The current flow rate may then be adjusted to meet the new target oxygen flow rate. The new target oxygen flow rate may then be verified by measuring a new current oxygen flow rate.

At block S712, the method 700 includes for adjusting the first flow rate with the flow controller to bring the predicted oxygen saturation level within a threshold value of the target oxygen saturation level. In some embodiments, the threshold value is about 5% to about 0.5% of the target oxygen saturation level. In other embodiments, the threshold value is about 2.5% to about 0.5% of the target oxygen saturation level. In further embodiments, the threshold value is about 1% to about 0.5% of the target oxygen saturation level. In some embodiments, the threshold value is about 5% of the target oxygen saturation level. In other embodiments, the threshold value is about 2.5% of the target oxygen saturation level. In further embodiments, the threshold value is about 1% of the target oxygen saturation level. In still further embodiments, the threshold value is about 0.5% of the target oxygen saturation level. In additional embodiments, the threshold value is less than about 0.5% of the target oxygen saturation level.

In many embodiments, blocks S708, S710, and S712 can be repeated continuously for the duration of the gas therapy as determined by a user input. In various embodiments, the above blocks can be repeated about once every minute to about once every half a second. In other embodiments, they can be repeated about once every thirty seconds to about once every half a second. In further embodiments, they can be repeated about once every five seconds to about once every half a second. In still further embodiments, they can be repeated about once every second to about once every half a second. In additional embodiments, they can be repeated about once every minute, about once every thirty seconds, about once every five seconds, about once every second, about once every half a second, or as fast as a processor of the device is capable, which for modern computing devices is many times a second and equivalent to a “live” update.

At optional step S714, the method 700 optionally includes for comparing a measured oxygen saturation level (as received from a physiological sensor such as an SpO2 sensor) with the minimum oxygen saturation level. This block can be repeated continuously for the duration of the oxygen therapy. In various embodiments, the above block about once every minute to about once every half a second. In other embodiments, it can be repeated about once every thirty seconds to about once every half a second. In further embodiments, it can be repeated about once every five seconds to about once every half a second. In still further embodiments, it can be repeated about once every second to about once every half a second. In additional embodiments, it can be repeated about once every minute, about once every thirty seconds, about once every five seconds, about once every second, about once every half a second, or as fast as the processor and physiological sensor of the device is capable, which for modern computing devices is many times a second and equivalent to a “live” update.

At optional block S716, the method 700 includes for triggering the alarm when the measured oxygen saturation level is less than the minimum oxygen saturation level. The alarm thus alerts a user (e.g., a patient or a healthcare provider) that the patient's oxygen saturation level has slipped past the inputted minimum oxygen saturation level and that further assistance and care is required.

In some embodiments, when a patient no longer needs oxygen therapy, it can be advantageous to slowly wean him or her off the supplemental oxygen. In these embodiments, the method 700 can optionally further comprise a step of decreasing at least one of the target oxygen saturation level or flow rate over a second time window to introduce an unassisted physiological oxygen saturation level. In many embodiments, by adjusting the target oxygen saturation level alone, the method 700 will automatically adjust the first flow rate in order to bring the predicted oxygen saturation level within a threshold value of the new target oxygen saturation level as described herein. In other embodiments, the method 700 can further decrease the flow rate once a target oxygen saturation level has been reached to further encourage an unassisted physiological uptake of oxygen. In many of these embodiments, the method 700 can increase the flow rate again if necessary to maintain a patient at or above a predetermined minimum oxygen saturation level. This process can be considered an “auto-wean” or “wean mode” process. In many embodiments, various alarm conditions can trigger an alarm during the auto-wean process. In some embodiments, there are alarm conditions unique to the auto-wean process.

In some embodiments, the second time window can be from about twenty-four hours to about five minutes. In other embodiments, the second time window can be from about twenty-four hours to about ten minutes. In still other embodiments, the second time window can be from about twenty-four hours to about fifteen minutes. In further embodiments, the second window of time can be from about twenty-four hours to about three hours. In still further embodiments, the second time window can be from about twelve hours to about three hours. In additional embodiments, the second time window can be from about twelve hours to about six hours. In still additional embodiments, the second time window can be from about six hours to about three hours. In some embodiments, the second time window can be from about three hours to about five minutes. In other embodiments, the second time window can be from about one hour to about five minutes. In further embodiments, the second time window can be from about thirty minutes to about five minutes. In still further embodiments, the second time window can be about fifteen minutes.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor on the device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “sensor” may include, and is contemplated to include, a plurality of sensors. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A device for administering oxygen therapy to a patient, the device comprising:
a gas intake valve;
a primary flow path connecting the gas intake valve to a gas output connector, the primary flow path comprising a flow controller configured to adjust a first flow rate through the primary flow path, wherein the flow controller is in electronic communication with a processor and memory;
at least one physiological sensor communicatively coupled to the processor;
wherein the memory stores machine-readable instructions that, when executed by the processor, cause the processor to perform a method comprising:
receiving a target oxygen saturation level;
receiving at least one measured physiological parameter from the at least one physiological sensor;
analyzing the at least one measured physiological parameter to determine a predicted oxygen saturation level of a patient for a first time window; and
adjusting, based on the analyzing of the at least one physiological parameter, the first flow rate with the flow controller to bring the predicted oxygen saturation level within a threshold value of the target oxygen saturation level.

2. The device of claim 1, further comprising:
a bypass flow path connecting the gas intake valve to the gas output connector, wherein the bypass flow path comprises a manual flow rate adjuster.

3. (canceled)

4. (canceled)

5. (canceled)

6. The device of claim 2, wherein the primary flow path and the bypass flow path comprise a shared flowmeter, and wherein the primary flow path and the bypass flow path merge upstream of the shared flowmeter.

7. The device of claim 2, wherein the device is configured to deliver gas through the primary flow path under a first operating condition and through the bypass flow path under a second operating condition, wherein the device is configured to change from the first operating condition to the second operating condition when a bypass criterion is detected or inputted.

8. The device of claim 7, further comprising a bypass switch, wherein the bypass criterion is selected from the group consisting of: a loss of device power, a system error, or a user selection of an emergency bypass switch.

9. The device of claim 7, wherein the device is configured to change from the second operating condition to the first operating condition by a user input.

10. (canceled)

11. The device of claim 1, wherein the threshold value is about 5% to about 0.5% of the target oxygen saturation level.

12. (canceled)

13. The device of claim 1, wherein the at least one physiological sensor is selected from the group consisting of: an SpO2 sensor, an EtCO2 sensor, an accelerometer, a pressure sensor, a microphone, a heart rate monitor, a back pressure monitor, an SpO2 waveform monitor, and an EKG.

14. The device of claim 1, wherein the at least one measured physiological parameter is selected from the group consisting of: an oxygen saturation level, a rate of change of an oxygen saturation level, an EtCO2 level, a rate of change of an EtCO2 level, a respiratory rate, a rate of change of a respiratory rate, a heart rate, a rate of change of a heart rate, a motion of the patient, an activity level of the patient an audio signal of a patient breathing, a cyclic back pressure, and a chest rise monitoring.

15. The device of claim 1, further comprising at least one internal sensor.

16. The device of claim 15, wherein the at least one internal sensor is a humidity sensor and a pressure sensor.

17. The device of claim 1, wherein the first time window is about 1 second to about 5 hours.

18. (canceled)

19. (canceled)

20. (canceled)

21. (canceled)

22. (canceled)

23. (canceled)

24. (canceled)

25. The device of claim 1, wherein receiving the minimum oxygen saturation level further comprises receiving a user input of the minimum oxygen saturation level, and wherein receiving the target oxygen saturation level further comprises receiving a user input of the target oxygen saturation level.

26. (canceled)

27. The device of claim 1, wherein receiving the target oxygen saturation level further comprises:
receiving one or more user inputs; and
determining the target oxygen saturation level based on the one or more user inputs.

28. The device of claim 1, wherein the method performed by processor further comprises decreasing at least one of the target oxygen saturation level or first flow rate over a second time window down to an unassisted physiological level.

29. The device of claim 1, wherein the device further comprises an alarm, wherein the at least one measured physiological parameter comprises a measured oxygen saturation level, and wherein the method further comprises:
comparing the measured oxygen saturation level to the minimum oxygen saturation level; and
triggering the alarm when the measured oxygen saturation level is less than the minimum oxygen saturation.

30. The device of claim 1, wherein the device further comprises an alarm and wherein the method further comprises:
measuring a first respiratory rate with at least one usage dependent sensor;
measuring a second respiratory rate with at least one usage independent sensor; and
comparing the first respiratory rate with the second respiratory rate;
wherein when the first respiratory rate and second respiratory rate are not within a respiratory comparison threshold value, triggering at least one of an alarm or an instruction condition.

31. A device for administering oxygen therapy to a patient, the device comprising:
a gas intake valve;
a primary flow path connecting the gas intake valve to a gas output connector, the primary flow path comprising a flow controller configured to adjust a first flow rate through the primary flow path, wherein the flow controller is in electronic communication with a processor and memory;
a bypass flow path connecting the gas intake valve to the gas output connector, wherein the bypass flow path comprises a manual flow rate adjuster;
at least one flow meter in fluid communication with and downstream of the flow controller and the manual flow rate adjuster;
at least one physiological sensor communicatively coupled to the processor;
at least one alarm;
wherein the processor and memory store machine-readable instructions that, when executed by the processor, cause the processor to perform a method comprising:
receiving a target oxygen saturation level;
receiving a minimum oxygen saturation level;
receiving at least one measured physiological parameter from the at least one physiological sensor, wherein the at least one measured physiological parameter comprises a measured oxygen saturation level;
analyzing the at least one measured physiological parameter to determine a predicted oxygen saturation level of a patient for a first time window;
comparing the measured oxygen saturation level to the minimum oxygen saturation level and triggering the alarm when the measured oxygen saturation level is less than the minimum oxygen saturation; and
adjusting, based on the analyzing of the at least one measured physiological parameter, the first flow rate with the flow controller to bring the predicted oxygen saturation level within a threshold value of the target oxygen saturation level;
wherein the device is configured to deliver oxygen gas through the primary flow path under a first operating condition;
wherein the device is configured to deliver oxygen gas through the bypass flow path under a second operating condition; and
wherein the device is configured to change from the first operating condition to the second operating condition when an emergency criterion is detected or inputted.

32. A method for delivering oxygen therapy to a patient comprising:
providing a device comprising:
a gas intake valve;
a primary flow path connecting the gas intake valve to a gas output connector, the primary flow path comprising a flow controller configured to adjust a first flow rate through the primary flow path; and
at least one physiological sensor;
receiving a target oxygen saturation level;
receiving at least one measured physiological parameter from the at least one physiological sensor;
analyzing the at least one measured physiological parameter to determine a predicted oxygen saturation level of a patient for a first time window; and
adjusting the first flow rate with the flow controller to bring the predicted oxygen saturation level within a threshold value of the target oxygen saturation level.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. The method of claim 32, wherein the device further comprises an alarm and wherein the method further comprises:
measuring a first respiratory rate with at least one usage dependent sensor;
measuring a second respiratory rate with at least one usage independent sensor;
comparing the first respiratory rate with the second respiratory rate; and
wherein when the first respiratory rate and second respiratory rate are not within a respiratory comparison threshold value, triggering at least one of an alarm or an instruction condition.
